(19) United States
(12) Patent Application Publication
(10) Pub. No.: US 2023/0167395 A1
(43) Pub. Date: Jun. 1, 2023

(54) THERMOSTABLE PHYTASE FOR LOW SODIUM STARCH LIQUEFACTION

(71) Applicant: DANISCO US INC., PALO ALTO, CA (US)

(72) Inventor: MOHAMMAD CHEGENI, CEDAR RAPIDS, IA (US)

(21) Appl. No.: 17/916,652

(22) PCT Filed: Apr. 20, 2021
(86) PCT No.: PCT/US2021/028141

§ 371 Date: Oct. 3, 2022

Related U.S. Application Data
(60) Provisional application No. 63/012,323, filed on Apr. 20, 2020.

(51) Int. Cl. C12M 1/00; C12N 9/16; C12N 9/26

(52) U.S. Cl. CPC C12M 39/00; C12Y 301/03; C12N 9/16; C12N 9/2414

(57) ABSTRACT

Described is a method for reducing the amount of thermostable α-amylase required for starch liquefaction performed under low sodium conditions by supplementing liquefaction with a thermostable phytase.

### CROSS REFERENCE

This application is a 371 of International Application No. PCT/US2021/028141, filed Apr. 20, 2021 and claims benefit to U.S. Provisional Application No. 63/012323, filed Apr. 20, 2020, and are incorporated in their entirety by reference.

### FIELD OF THE INVENTION

Disclosed is a method for reducing the amount of thermostable α-amylase required for starch liquefaction performed under low sodium conditions by supplementing liquefaction with a thermostable phytase.

### BACKGROUND

Caustic wash down conditions are often used to clean liquefaction tanks and related equipment in dry grind milling facilities making fuel ethanol. A substantial portion of the wash down solution can remain in the tanks and becomes incorporated into subsequent liquefactions. Where sodium hydroxide is used for wash down, a significant amount of sodium can be added to the liquifaction.

Thermostable α-amylases are known to be stabilized by both calcium and sodium ions. Sodium introduced into liquefaction tanks via sodium hydroxide wash down contributes to the stability these enzymes during liquefaction. Accordingly, the use of non-caustic wash down agents introduces a challenge in terms of providing of a stabilizing environment for α-amylases, requiring the use of increased amount of the enzyme. While sodium can be added during liquefaction, this will typically require addition of an anions, which may adversely affect downstream processing.

The need exists to offset the reduced stability of α-amylases resulting from the from the use of non-caustic wash down solutions.

### SUMMARY

Disclosed is a method for reducing the amount of thermostable α-amylase required for starch liquefaction under low sodium conditions by supplementing liquefaction with a thermostable phytase. Aspects and embodiments of the present compositions and methods are summarized in the following, separately-numbered paragraphs:

1. In one aspect, in a method for washing down internal surfaces of a dry grind ethanol facility using acids and/or peracids to replace sodium hydroxide, an improvement is provided comprising adding during liquefaction an amount of thermostable phytase sufficient to offset the increase in the amount of α-amylase required by the reduced amount of sodium present during liquefaction.

2. In a related aspect, in a method for washing down internal surfaces of a dry grind ethanol facility using acids and/or peracids, an improvement is provided comprising adding to starch liquefaction an amount of thermostable phytase sufficient to offset the reduction in thermostable α-amylase stability compared to the thermostable α-amylases stability in an otherwise identical starch liquefaction in a facility using caustic sodium hydroxide for wash down.

3. In some embodiments of the method of paragraph 1 or 2, the acids and/or peracids are selected from the group consisting of phosphoric acid, formic acid, acetic acid, octanoic acid, peroxyacetic acid, peroxyoctanoic acid and combinations, thereof.

4. In some embodiments of the method of any of paragraphs 1-3, the thermostable phytase is derived from an organism selected from the group consisting of a Buttiauxella sp., a Citrobacter sp, an Escherichia sp., a Peniophora sp. or an Obesumbacterium sp.

5. In some embodiments of the method of any of paragraphs 1-3, the thermostable phytase is AXTRA® PHY or RONOZYME®.

6. In some embodiments of the method of any of paragraphs 1-5, the thermostable α-amylases is derived from an organism selected from the group consisting of Bacillus stearothermophilus, B. licheniformis, B. amyloliquifaciens, a Cytophaga sp., or from a hybrid molecule, thereof.

7. In some embodiments of the method of any of paragraphs 1-5, the thermostable α-amylases is SPEZYME®-AA, SPEZYME®-Alpha, SPEZYME®-Ethyl, SPEZYME®-Fred, SPEZYME®-Xtra and SPEZYME®-RSL, CLARASE™ L, GZYME™ 997, GC356, TERMAMYL™ 120-L, TERMAMYL™ LC, TERMAMYL™ SC, TERMAMYL™ SUPRA, LIQUOZYME™ X, SAN™ SUPER, LPHERA® FORTIVA® and FUELZYME™ LF.

8. In some embodiments of the method of any of paragraphs 1-7, liquefaction is performed in the presence of a protease.

9. In some embodiments of the method of any of paragraphs 1-7, liquefaction is performed in the presence of DCO+® or AVENTEC® AMP.

These and other aspects and embodiments of the present compositions and methods will be apparent from the following description and appended Examples.

### DETAILED DESCRIPTION

### 1. Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the enzyme” includes reference to one or more enzymes and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are defined, below, for clarity.

### 1.1. Abbreviations and Acronyms

The following abbreviations/acronyms have the following meanings unless otherwise specified:

° C. degrees Centigrade

dH₂O or DI deionized water

g or gm grams

hr(s) hour/hours

kg kilograms

M molar

mg milligrams

min(s) minute/minutes

mL and ml milliliters

mm millimeters

mM millimolar

MW molecular weight

sec seconds

U units

v/v volume/volume

w/v weight/volume

w/w weight/weight

wt % weight percent

### 1.2. Definitions

As used herein the term “starch” refers to any material comprised of the complex polysaccharide carbohydrates of plants, comprised of amylose and/or amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum, legumes, cassava, millet, potato, sweet potato, and tapioca. After purification of the complex polysaccharide carbohydrates from the other plant components, it is called “refined starch.”

As used herein, the term “phytase” refers to a protein capable of catalyzing the hydrolysis of phytate (phytic acid) to inositol and phosphate or to mono-, di-, tri-, tetra- and/or penta-phosphates of inositol and inorganic phosphate. Phytases have the Enzyme Commission EC numbers 3.1.3.8 and 3.1.3.26.

As used herein, the term “α-amylase” refers to an enzyme that is, among other things, capable of catalyzing the degradation of starch. α-amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) O-glycosidic linkages within the starch molecule in a random fashion yielding polysaccharides containing three or more (1-4)-α-linked D-glucose units.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t_{1/2}) given in minutes, hours, or days, during which half the enzyme activity is lost under defined conditions. The half-life may be calculated by measuring residual α-amylase activity following exposure to (i.e., challenge by) an elevated temperature.

The term “specific activity” refers to the number of moles of substrate that can be converted to product by an enzyme or enzyme preparation per unit time under specific conditions. Specific activity is generally expressed as units (U)/mg of protein.

### 2. Thermostable Phytase for Low Sodium Starch Liquefaction

Dry grind ethanol mills often use caustic solutions to wash down tanks and equipment. Where liquefaction tanks are washed down with sodium hydroxide, some amount of wash down solution remains in the tanks and contributes sodium to subsequent liquefactions. Thermostable α-amylases used in starch liquefaction are stabilized by the sodium contributed by the wash down solution.

As an alternative to caustic solutions such as sodium hydroxide, some dry grind ethanol mills use acids and peracids, such as phosphoric acid, formic acid, acetic acid, octanoic acid, peroxyacetic acid, peroxyoctanoic acid and combinations, thereof. While these wash down solutions may offer some advantages over caustics, they generally do not contribute sodium to liquefactions, which can mean that additional α-amylase is required to achieve the same degree of starch liquefaction.

The present method addresses the issue of reduced α-amylase stability under reduced sodium conditions by way of addition of thermostable phytase to liquefaction. Phytase is known to hydrolyzes phytic acid, which is an inhibitor of α-amylases. Therefore, it is known that phytase activity indirectly increases the activity of α-amylase. However, it was heretofore unknown that phytase would offset the loss in stability of α-amylase in liquifaction performed under low sodium conditions.

The thermostable phytase may be added to liquifaction prior to adding the thermostable α-amylase, simultaneously with adding the α-amylase, or shortly after adding the α-amylase. Preferrably, the phytase is added simultaneously α-amylase in the form of multi-enzyme composition. A commercially available example of such a product is SPEZYME®-RSL, which contains an engineered Bacillus α-amylase and an engineered as Buttiauxella phytase.

In some embodiments, other thermostable enzymes are added to liquifaction, including proteases.

3. Enzymes for Use in Low Sodium Liquefaction

Commercially-available α-amylases for use in low sodium liquifaction include but are not limited to thermostable α-amylases that have been previously described for use in wet and dry grind milling. Such enzymes include bacterial enzymes, such as SPEZYME®-AA, SPEZYME®-Alpha, SPEZYME®-Ethyl, SPEZYME®-Fred, SPEZYME®-Xtra and SPEZYME®-RSL, CLARASE™ L, GZYIVIE™ 997 and GC356 (DuPont), TERMAMYL™ 120-L, TERMAMYL™ LC and TERMAMYL™ SC and SUPRA, LIQUOZYIVIIE™ X, SAN™ SUPER, LPHERA® and FORTIVA® (Novozymes A/S), and FUELZYME™ LF (Diversa). In some embodiments, the thermostable α-amylase will be derived from Bacillus stearothermophilus, B. licheniformis, B. amyloliquifaciens, a Cytophaga sp., or from a hybrid molecules based on one or more of these enzymes or other enzymes. Commercially-available thermostable fungal amylases include GC626® (DuPont) from Aspergillus kawachii.

Commercially-available thermostable phytase enzymes include AXTRA® PHY (DuPont) and RONOZYME® (Novozymes). In some embodiments, the thermostable phytase will be derived from an organism such as Buttiauxella sp., a Citrobacter sp, an Escherichia sp., a Peniophora sp. or an Obesumbacterium sp.

Commercially-available thermostable protease enzymes include DCO+® (DuPont) and AVENTEC® AMP (Novozymes). In some embodiments, the thermostable protease will be derived from an organism such as a Thermobifida sp., a Nocardiopsis sp., a Thermococcus sp. a Streptomyces sp. or a Pyrococcus sp. A classic thermostable protease is thermolysin, a neutral metalloproteinase produced by the Gram-positive bacteria Bacillus thermoproteolyticus.

All references cited herein are herein incorporated by reference in their entirety for all purposes.

1. In a method for washing down internal surfaces of a dry grind ethanol facility using acids and/or peracids to replace sodium hydroxide, an improvement comprising adding during liquefaction an amount of thermostable phytase sufficient to offset the increase in the amount of α-amylase required by the reduced amount of sodium present during liquefaction.

2. In a method for washing down internal surfaces of a dry grind ethanol facility using acids and/or peracids, an improvement comprising adding to starch liquefaction an amount of thermostable phytase sufficient to offset the reduction in thermostable α-amylase stability compared to the thermostable α-amylases stability in an otherwise identical starch liquefaction in a facility using caustic sodium hydroxide for wash down.

3. The method of claim 1 or 2, wherein the acids and/or peracids are selected from the group consisting of phosphoric acid, formic acid, acetic acid, octanoic acid, peroxyacetic acid, peroxyoctanoic acid and combinations, thereof.

4. The method of any of claims 1-3, wherein the thermostable phytase is derived from an organism selected from the group consisting of a Buttiauxella sp., a Citrobacter sp, an Escherichia sp., a Peniophora sp. or an Obesumbacterium sp.

5. The method of any of claims 1-3, wherein the thermostable phytase is AXTRA® PHY or RONOZYME®.

6. The method of any of claims 1-5, wherein the thermostable α-amylases is derived from an organism selected from the group consisting of Bacillus stearothermophilus, B. licheniformis, B. amyloliquifaciens, a Cytophaga sp., or from a hybrid molecule, thereof.

7. The method of any of claims 1-5, wherein the thermostable α-amylases is SPEZYME®-AA, SPEZYME®-Alpha, SPEZYME®-Ethyl, SPEZYME®-Fred, SPEZYME®-Xtra and SPEZYME®-RSL, CLARASE™ L, GZYME™ 997, GC356, TERMAMYL™ 120-L, TERMAMYL™ LC, TERMAMYL™ SC, TERMAMYL™ SUPRA, LIQUOZYME™ X, SAN™ SUPER, LPHERA® FORTIVA® and FUELZYME™ LF.

8. The method of any of claims 1-7, wherein liquefaction is performed in the presence of a protease.

9. The method of any of claims 1-7, wherein liquefaction is performed in the presence of DCO+® or AVENTEC® AMP.
